SEQUENCE LISTING (72 sequences)

SEQ ID NO: 1 - RNA, 22 nt, Artificial Sequence
  source: /note=“Description of Artificial Sequence Synthetic oligonucleotide”
gggagacgcguguuaaauaaca

SEQ ID NO: 2 - DNA, 22 bp, Artificial Sequence
  source: /note=“Description of Artificial Sequence Synthetic oligonucleotide”
ggatccggaggccggagaattg

SEQ ID NO: 3 - DNA, 17 bp, Artificial Sequence
  source: /note=“Description of Artificial Sequence Synthetic oligonucleotide”
taatacgactcactata

SEQ ID NO: 4 - RNA, 14 nt, Artificial Sequence
  source: /note=“Description of Artificial Sequence Synthetic oligonucleotide”
ccugaaaaaaaaaa

SEQ ID NO: 5 - RNA, 24 nt, Artificial Sequence
  source: /note=“Description of Artificial Sequence Synthetic oligonucleotide”
ccugaaaaaaaaaaaaaaaaaaaa

SEQ ID NO: 6 - DNA, 27 bp, Artificial Sequence
  source: /note=“Description of Artificial Sequence Synthetic oligonucleotide”
aaaaaaaaaaaaaaaaaaaaaaaaaaa

SEQ ID NO: 7 - DNA, 100 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 8 - DNA, 117 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 9 - DNA, 10 bp, Artificial Sequence
  source: /note=“Description of Artificial Sequence Synthetic oligonucleotide”
aaaaaaaaaa

SEQ ID NO: 10 - DNA, 20 bp, Artificial Sequence
  source: /note=“Description of Artificial Sequence Synthetic oligonucleotide”
aaaaaaaaaaaaaaaaaaaa

SEQ ID NO: 11 - DNA, 64 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaa

SEQ ID NO: 12 - DNA, 120 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 13 - DNA, 117 bp, Artificial Sequence; identical to SEQ ID NO: 8
  source: /note=“Description of Artificial Sequence Synthetic polynucleotide”
  misc_feature (1)..(117): /note=“This sequence may encompass 27-117 nucleotides”

SEQ ID NO: 14 - DNA, 25 bp, Artificial Sequence
  source: /note=“Description of Artificial Sequence Synthetic oligonucleotide”
ttttttttttttttttttttttttt

SEQ ID NO: 15 - DNA, 109 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 16 - DNA, 110 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 111 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 18 - DNA, 112 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 113 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 20 - DNA, 114 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 21 - DNA, 115 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 22 - DNA, 116 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 23 - DNA, 118 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 24 - DNA, 119 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 25 - DNA, 121 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - DNA, 122 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 27 - DNA, 62 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaa

SEQ ID NO: 28 - DNA, 63 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaa

SEQ ID NO: 29 - DNA, 65 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaa

SEQ ID NO: 30 - DNA, 66 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaa

SEQ ID NO: 31 - DNA, 67 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaa

SEQ ID NO: 32 - DNA, 68 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaa

SEQ ID NO: 33 - DNA, 69 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaa

SEQ ID NO: 34 - DNA, 70 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaa

SEQ ID NO: 35 - DNA, 71 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaa

SEQ ID NO: 36 - DNA, 72 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaa

SEQ ID NO: 37 - DNA, 73 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaa

SEQ ID NO: 38 - DNA, 74 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaa

SEQ ID NO: 39 - DNA, 75 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaa

SEQ ID NO: 40 - DNA, 76 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaaa

SEQ ID NO: 41 - DNA, 95 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 42 - DNA, 96 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 43 - DNA, 97 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 44 - DNA, 98 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 45 - DNA, 99 bp, Artificial Sequence
  source: /note=“Description of Artificial Sequence Synthetic oligonucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 46 - DNA, 101 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 47 - DNA, 102 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 48 - DNA, 103 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 49 - DNA, 104 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - DNA, 105 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 51 - DNA, 106 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 52 - DNA, 107 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 53 - DNA, 108 bp, Artificial Sequence
  source: /note=“Description of Artificial Sequence Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 54 - DNA, 26 bp, Artificial Sequence
  source: /note=“Description of Artificial Sequence Synthetic oligonucleotide”
aaaaaaaaaaaaaaaaaaaaaaaaaa

SEQ ID NO: 55 - DNA, 28 bp, Artificial Sequence
  source: /note=“Description of Artificial Sequence Synthetic oligonucleotide”
aaaaaaaaaaaaaaaaaaaaaaaaaaaa

SEQ ID NO: 56 - DNA, 29 bp, Artificial Sequence
  source: /note=“Description of Artificial Sequence Synthetic oligonucleotide”
aaaaaaaaaaaaaaaaaaaaaaaaaaaaa

SEQ ID NO: 57 - DNA, 30 bp, Artificial Sequence
  source: /note=“Description of Artificial Sequence Synthetic oligonucleotide”
aaaaaaaaaaaaaaaaaaaaaaaaaaaaaa

SEQ ID NO: 58 - DNA, 31 bp, Artificial Sequence
  source: /note=“Description of Artificial Sequence Synthetic oligonucleotide”
aaaaaaaaaaaaaaaaaaaaaaaaaaaaaaa

SEQ ID NO: 59 - DNA, 32 bp, Artificial Sequence
  source: /note=“Description of Artificial Sequence Synthetic oligonucleotide”
aaaaaaaaaaaaaaaaaaaaaaaaaaaaaaaa

SEQ ID NO: 60 - DNA, 33 bp, Artificial Sequence
  source: /note=“Description of Artificial Sequence Synthetic oligonucleotide”
aaaaaaaaaaaaaaaaaaaaaaaaaaaaaaaaa

SEQ ID NO: 61 - DNA, 34 bp, Artificial Sequence
  source: /note=“Description of Artificial Sequence Synthetic oligonucleotide”
aaaaaaaaaaaaaaaaaaaaaaaaaaaaaaaaaa

SEQ ID NO: 62 - DNA, 35 bp, Artificial Sequence
  source: /note=“Description of Artificial Sequence Synthetic oligonucleotide”
aaaaaaaaaaaaaaaaaaaaaaaaaaaaaaaaaaa

SEQ ID NO: 63 - DNA, 36 bp, Artificial Sequence
  source: /note=“Description of Artificial Sequence Synthetic oligonucleotide”
aaaaaaaaaaaaaaaaaaaaaaaaaaaaaaaaaaaa

SEQ ID NO: 64 - DNA, 37 bp, Artificial Sequence
  source: /note=“Description of Artificial Sequence Synthetic oligonucleotide”
aaaaaaaaaaaaaaaaaaaaaaaaaaaaaaaaaaaaa

SEQ ID NO: 65 - DNA, 38 bp, Artificial Sequence
  source: /note=“Description of Artificial Sequence Synthetic oligonucleotide”
aaaaaaaaaaaaaaaaaaaaaaaaaaaaaaaaaaaaaa

SEQ ID NO: 66 - DNA, 39 bp, Artificial Sequence
  source: /note=“Description of Artificial Sequence Synthetic oligonucleotide”
aaaaaaaaaaaaaaaaaaaaaaaaaaaaaaaaaaaaaaa

SEQ ID NO: 67 - DNA, 40 bp, Artificial Sequence
  source: /note=“Description of Artificial Sequence Synthetic oligonucleotide”
aaaaaaaaaaaaaaaaaaaaaaaaaaaaaaaaaaaaaaaa

SEQ ID NO: 68 - DNA, 41 bp, Artificial Sequence
  source: /note=“Description of Artificial Sequence Synthetic oligonucleotide”
aaaaaaaaaaaaaaaaaaaaaaaaaaaaaaaaaaaaaaaaa

SEQ ID NO: 69 - DNA, 42 bp, Artificial Sequence
  source: /note=“Description of Artificial Sequence Synthetic oligonucleotide”
aaaaaaaaaaaaaaaaaaaaaaaaaaaaaaaaaaaaaaaaaa

SEQ ID NO: 70 - DNA, 43 bp, Artificial Sequence
  source: /note=“Description of Artificial Sequence Synthetic oligonucleotide”
aaaaaaaaaaaaaaaaaaaaaaaaaaaaaaaaaaaaaaaaaaa

SEQ ID NO: 71 - DNA, 44 bp, Artificial Sequence
  source: /note=“Description of Artificial Sequence Synthetic oligonucleotide”
aaaaaaaaaaaaaaaaaaaaaaaaaaaaaaaaaaaaaaaaaaaa

SEQ ID NO: 72 - DNA, 45 bp, Artificial Sequence
  source: /note=“Description of Artificial Sequence Synthetic oligonucleotide”
aaaaaaaaaaaaaaaaaaaaaaaaaaaaaaaaaaaaaaaaaaaaa

(19) United States
(12) Patent Application Publication
(10) Pub. No.: US 2020/0325532 A1
(43) Pub. Date: Oct. 15, 2020

(54) POLYA TAIL LENGTH ANALYSIS OF RNA BY MASS SPECTROMETRY

(71) Applicant: Novartis AG, Basel (CH)

(72) Inventors: Michael BEVERLY, Lexington, MA (US); Caitlin Jeanette HAGEN, (US); Olga SLACK, Newton, MA (US)

(21) Appl. No.: 16/772,859

(22) PCT Filed: Dec. 14, 2018
(86) PCT No.: PCT/IB2018/060121

§ 371 Date: Jun. 15, 2020

Related U.S. Application Data
(60) Provisional application No. 62/599,205, filed on Dec. 15, 2017.

(51) Int. Cl. C12Q 1/6844

(52) U.S. Cl. CPC C12Q 1/6844; C12Q 2521/107; C12Q 2565/627; C12Q 2563/149; C12Q 2563/143; C12Q 2521/327

(57) ABSTRACT

Described herein are methods of analyzing and producing RNA compositions, e.g., mRNA compositions, that include determining the amount of a polyA chain length and/or 5 the relative distribution of polyA chain lengths in a sample from the RNA composition using mass spectrometry, e.g., LC-MS or MALDI-MS.

### CROSS-REFERENCE TO RELATED APPLICATION

This application claims the priority and the benefit of U.S. Patent Application No. 62/599,205, filed Dec. 15, 2017, the contents of which are incorporated herein by their entireties.

### GOVERNMENT INTERESTS

This invention was made with government support under contract number HR0011-13-3-0003, awarded by the Defense Advanced Research Projects Agency (DARPA). The government has certain rights in the invention.

### REFERENCE TO A “SEQUENCE LISTING,” A TABLE, OR A COMPUTER PROGRAM LISTING APPENDIX SUBMITTED AS AN ASCII FILE

The Sequence Listing written in file PAT058018-WO-PCT_SL.TXT, created Dec. 11, 2018, 22,449 bytes in size, machine format IBM-PC, MS-Windows operating system, is hereby incorporated by reference.

### FIELD OF THE INVENTION

The disclosure relates to methods of analyzing compositions of RNA (e.g., mRNA), e.g., for suitability for use as a therapeutic or for use in making an RNA therapeutic, using mass spectrometry, e.g., liquid chromatography coupled to electrospray mass spectrometry (LC-MS) or matrix-assisted laser desorption/ionization mass spectrometry (MALDI-MS), to determine the polyA species present in the RNA composition. In particular, the methods can be used to evaluate compositions of RNA and specifically to methods of evaluating compositions of messenger RNA (mRNA) having a polyA tail.

### BACKGROUND OF THE INVENTION

Eukaryotic mRNAs often terminate with a stretch of adenosines on their 3′ end. These 3′ polyadenosine (polyA) tails can vary in length from 20 to over 200 bases, depending on the species. The polyA tail is necessary for the translation and stability of the mRNA in the cytoplasm, through its interaction with polyA binding protein (PABP), the 5′ cap and the eukaryotic initiation factors (eIF).

Some have postulated that longer tails increase translation efficiency, provide mRNA stability, and can possibly affect immune response, although there has been some recent research suggesting that tail length of mRNA and translation efficiency are linked only during specific cell development stages. See, Bernstein et al., Mol. Cell Biol. 9: 659-670 (1989); Bernstein & Ross, Trends Biochem. Sci. 14: 373-377 (1989); Koski et al., J. Immunol. 172: 3989-3993 (2004); Beilharz & Preiss, RNA 13: 982-997 (2007); Peng et al., Methods Mol. Biol. 419: 215-230 (2008); Subtelny et al., Nature 508: 66-71 (2014); and Park et al., Mol. Cell 62: 462-471 (2016). For therapeutic mRNA that produces a specific therapeutic protein or immune stimulating antigen, the longevity and immunogenicity of the mRNA importantly influences the duration or intensity or both of protein expression.

Understanding polyA tail length and how it relates to mRNA stability and protein expression requires methods that accurately characterize the polyA tail length of IVT synthesized mRNA.

### BRIEF SUMMARY OF THE INVENTION

Provided herein is a method of evaluating the quality of an mRNA composition, including the steps of: (a) providing an evaluation by mass spectrometry of the relative distribution of isolated polyA chain lengths or amount of a polyA chain length that have been cleaved from the mRNA of a sample obtained from the mRNA composition, to provide a test value and (b) providing a determination of whether test value has an relative distribution or amount of a reference value, to thereby evaluate the quality of the mRNA composition.

In some embodiments, the method further comprises performing the mass spectrometry to determine the relative distribution of the isolated polyA chain lengths or the amount of a poly A chain length that have been cleaved from the mRNA of the sample.

In some embodiments, the mass spectrometry is LC-MS.

In some embodiments, the method further comprises providing a sample from the mRNA composition and cleaving the polyA tails from the mRNA in the sample using an enzyme or combination of enzymes that do not cleave adenosine.

In some embodiments, the enzyme is ribonuclease T1, RNAse CL3 (cusativin), RNase A or any combination thereof.

In some embodiments, the method further comprises isolating the cleaved polyA tails from the mRNA in the sample by hybridizing the cleaved polyA tails to a surface coated substrate conjugated with polynucleotides.

In some embodiments, the surface coated substrate is a magnetic bead.

In some embodiments, the magnetic bead is conjugated with oligo dT.

In some embodiments, the mRNA is made by in vitro transcription (IVT).

Also provided herein is a radiolabel-free method for analyzing the 3′-polyadenosine (polyA) tails of mRNA in an mRNA composition, the method includes the steps of (a) cleaving polyA tails from a sample of the RNA composition using ribonuclease T1, RNAse CL3 (cusativin), RNase A or any combination thereof; (b) isolating the cleaved polyA tails by hybridization to surface coated substrate that are conjugated to a polynucleotide; and (c) determining the relative distribution of poly A chain lengths or the amount of a polyA chain in the sample using mass spectrometry, to thereby analyze the polyA tails present in the mRNA composition.

In some embodiments, the mass spectrometry is LC-MS.

In some embodiments, the method further includes providing a test value based upon the relative distribution of the polyA chain lengths or amount of the polyA chain in the sample and comparing the test value to a reference value.

In some embodiments, the surface coated substrate comprises magnetic beads.

In some embodiments, the polynucleotide that is conjugated to the surface coated substrate is oligo dT.

In some embodiments, the mRNA is made by in vitro transcription (IVT).

In some embodiments, the method is completed within five hours.

In some embodiments, the polyA tails within the composition range from ˜20 A's to ˜300 A's in length or longer.

Further provided herein is a method of making an RNA composition, e.g., an mRNA composition, the method includes: (a) providing an RNA sample from the RNA composition; (b) providing a relative distribution of polyA chain lengths or an amount of a polyA chain length from isolated polyA tails from the RNA in the RNA sample, by mass spectrometry, e.g., LC-MS or MALDI-MS, to provide a test value; (c) providing a determination of whether the test value is an amount or has a relative distribution of a reference value; and (d) further processing the RNA composition based upon the determination.

In some embodiments, the further processing is one or more of classifying, selecting, accepting or discarding, releasing or withholding, processing into a drug product, shipping, moving to a different location, formulating, labeling, packaging, releasing into commerce, or selling or offering for sale, based upon whether a preselected relationship between the test value and the reference value is met.

In some embodiments, the RNA sample has an amount of a polyA chain length or has a profile of polyA chain length distribution of a reference value, and the RNA composition is processed into drug product, formulated, labeled, packaged, or released into commerce based upon the determination.

In some embodiments, the RNA sample has an amount of a polyA chain length or has a profile of polyA chain length distribution of a reference value and the production method used to make the RNA composition is used to make an additional batch of the RNA composition.

In some embodiments, the RNA sample does not have an amount of a polyA chain length or does not have a profile of polyA chain length distribution of a reference value, and the RNA composition is discarded or withheld.

In some embodiments, the RNA sample does not have an amount of a polyA chain length or does not have a profile of polyA chain length distribution of a reference value, and the production method used to make the RNA composition is modified.

In some embodiments, the production method is modified by one or more of modifying the amount of ATPase used the production process of the RNA composition, e.g., increasing or decreasing, in subsequent batches of the RNA composition and modifying the amount of ATP used in the production process, e.g., increasing or decreasing, in subsequent batches of the RNA composition.

In some embodiments, the method further comprises cleaving the polyA tails from the mRNA in the sample using an enzyme or combination of enzymes that do not cleave adenosine.

In some embodiments, the enzyme is ribonuclease T1, RNAse CL3 (cusativin), RNase A or any combination thereof.

In some embodiments, the method further comprises isolating the cleaved polyA tails from the mRNA in the sample by hybridizing the cleaved polyA tails to a surface coated substrate conjugated with polynucleotides.

In some embodiments, the surface coated substrate is a magnetic bead.

In some embodiments, the magnetic bead is conjugated with oligo dT.

In some embodiments, the method further comprises producing the mRNA composition using IVT.

In some embodiments, the polyA tails of the mRNA composition are part of the DNA template for IVT.

In some embodiments, the poly A tails are enzymatically added to the IVT produced mRNA in the mRNA composition.

In some embodiments, the reference value is a value determined from an RNA sample from a commercially available RNA composition.

In some embodiments, the reference value is a value determined from a previous batch of the RNA composition.

In some embodiments, the reference value is or comprises a production standard imposed by a regulatory agency.

In some embodiments, the reference value is or comprises a release standard.

### BRIEF DESCRIPTION OF THE DRAWINGS

FIG. 1 is a set of chromatograms showing LC-MS analysis of two methods of making in vitro transcribed polyA tailed RNA. The top chromatogram shows that the enzymatically tailed mRNA has a much wider peak, resulting from a larger dispersity of polyA tail lengths, than the bottom chromatogram, which measured mRNA with plasmid-encoded polyA tails.

FIG. 2 is an image of a poly-acrylamide gel electrophoretogram, showing the length and integrity of 2100nt RNA with 27 A's (SEQ ID NO: 6), 100 A's (SEQ ID NO: 7) and 117 A's (SEQ ID NO: 8), each plasmid-encoded.

FIG. 3 is a chromatogram showing LC-MS analysis of a 10 mer and 20 mer polyA test sequence (SEQ ID NOS 9 and 10). Equal amounts of each polyA test sequence were measured by the polyA analysis method of T1 cleavage, followed by isolation of the polyA tail on oligo dT 25 mer (SEQ ID NO: 14) magnetic beads. The resulting total ion chromatogram from the mass spectrometer shows the T1 cleaved products for the 10 and 20 mer polyA sequences (SEQ ID NOS 9 and 10). The presence of single peaks for each polyA sequence shows that digestion was complete after 3 hr. FIG. 3 shows that the 10 mer is recovered less efficiently than the 20 mer.

FIG. 4 is a set of three UV250 nm chromatograms from the LC-MS analysis of mRNA with polyA tails of 100 (SEQ ID NO: 7), 64 (SEQ ID NO: 11) and 27 A's (SEQ ID NO: 6), respectively. At the 27 mer length (the bottom chromatogram), each individual peak represents a single tail species differing by one adenosine. This single nucleotide resolution is lost at the 64 mer and the 100 mer lengths, where peaks coalesce.

FIG. 5 is the resulting deconvoluted electrospray mass spectrum of the single peak in the chromatogram for the 100 mer tail length shown in FIG. 4. Mass versus spectral intensity is plotted. The series of peaks separated by the mass of adenosine represents the different polyA tail lengths observed. The peak at mass 33,163.9 (outlined in the box) represents the mass of a plasmid-encoded 100 mer polyA tail (SEQ ID NO: 7).

FIG. 6 is a graph of average mass spectral signal intensity versus polyA tail length for 3 separate T1 digestions of an mRNA with a polyA tail having a length of 117 A's (SEQ ID NO: 8). FIG. 6 also discloses SEQ ID NOS 15-22, 23-24, 12 and 25-26, respectively, in order of appearance.

FIG. 7 is a graph of average mass spectral signal intensity versus polyA tail length for an mRNA with a polyA tail having a length of 64 A's (SEQ ID NO: 11). FIG. 7 also discloses SEQ ID NOS 27-40, respectively, in order of appearance.

FIG. 8 is a chromatogram of polyA tails of an mRNA composition made by IVT from plasmids encoding 100 mer polyA tails (SEQ ID NO: 7). Minor populations of DNA templates each containing different polyA tail lengths were transcribed into the mRNA and the tail lengths observed by LC-MS closely matched the adenosine lengths found in the sequencing traces. FIG. 8 also discloses SEQ ID NOS 41-53, 15-16 and 19-22, respectively, in order of appearance.

FIG. 9 is a chromatogram of polyA tails of an mRNA composition made by IVT from plasmids encoding 27 mer tails. The measured tail lengths were longer than expected from the plasmid. FIG. 9 discloses SEQ ID NOS 54, 6 and 55-72, respectively, in order of appearance.

### DETAILED DESCRIPTION OF THE INVENTION

### Introduction

In vivo, the mRNA 3′ polyA tail affects mRNA function in several ways. In vivo, polyA tails of mRNA are produced through specific exonuclease trimming of the 3′ end followed by poly (A) polymerase.

mRNAs can be produced synthetically through in vitro transcription (IVT). In vitro, mRNAs gain their polyA tails either by encoding the polyA sequence into the template DNA or by having the polyA's added post-synthesis using a polyadenylase. The methods described herein can include a step of producing an mRNA composition, e.g., by either of these methods.

Measurement of the tail length in IVT synthesized mRNA can help in understanding how polyA tail length is related to mRNA function. Understanding polyA tail length and how it relates to mRNA stability and protein expression requires methods to accurately characterize the polyA tail length of IVT synthesized mRNA.

Assessing the heterogeneity of polyA tails in IVT mRNA can also be important from a clinical standpoint. mRNA is increasingly being produced for therapeutic purposes and having a consistent and well-defined product is important to reproducible activity.

There are many techniques to characterize the polyA tail length of mRNA, such as RNase H cleavage, chromatographic methods, PCR based assays like LM-PAT and ePAT, and next generation sequencing methods such as TAIL-seq and PAL-seq. See, e.g., Salles et al., PCR Methods Appl. 4: 317-321 (1995); Meijer et al., Nucleic Acids Res. 35: e132 (2007); Murray & Schoenberg, Methods EnzymoL 448: 483-504 (2008); Janicke et al., RNA 18: 1289-1295 (2012); Chang et al., Mol. Cell 53: 1044-1052 (2014); Subtelny et al., 2014. Nature 508: 66-71. Next generation sequencing approaches have the highest resolution (1 base reported for TAIL-seq), whereas those that use gels or chromatography usually show smears for a population of different tail lengths. However, PCR-based techniques can be complicated by the indirect nature of their measurement, which must amplify long stretches of adenosines.

Unlike other methods, mass spectrometry is a direct measurement technique that does not require labels and has the ability to distinguish between single nucleotides by their differences in mass. The methods described herein use mass spectrometry to study mRNA polyA tail length distributions. Mass spectrometry can provide information about the base composition of known PCR amplified sequence, which allows single nucleotide changes to be identified and multiple PCR products to be distinguished from one another. Furthermore, the methods described herein use mass spectrometry to directly measure many oligonucleotide sequences simultaneously with single nucleotide resolution. The results described in the EXAMPLES used standard LC-MS conditions for oligonucleotides to measure polyA chain lengths within an mRNA composition. The deconvolution of the resulting multiply charged spectra was done by a common software procedure in MS systems.

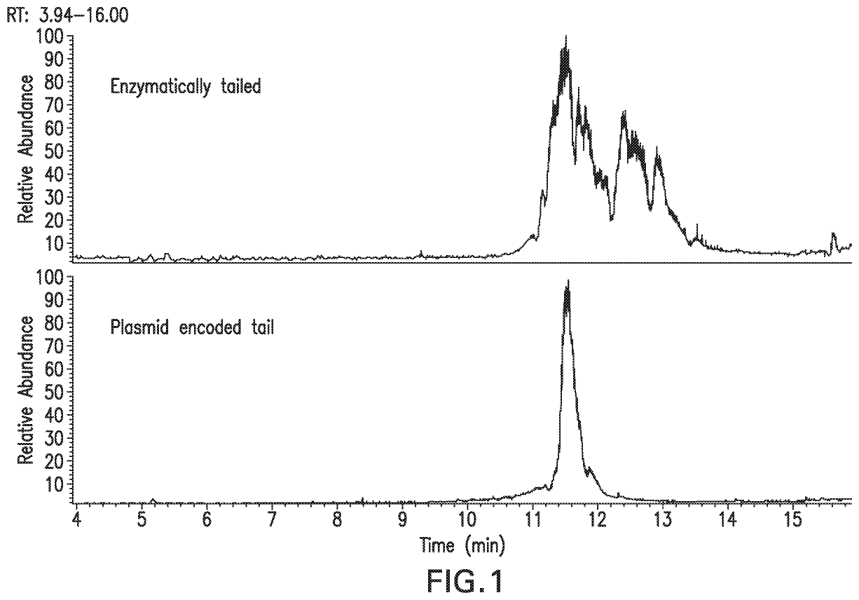

As described in EXAMPLE 1, 2100nt mRNAs with plasmid-encoded tail lengths of 27 (SEQ ID NO: 6), 64 (SEQ ID NO: 11), 100 (SEQ ID NO: 7) or 117 polyA's (SEQ ID NO: 8) were assayed. The results showed that enzymatically tailed mRNA have significant tail length heterogeneity. See, FIG. 1. The methods described herein measured the number of A's in the polyA tails and the numbers closely matched the expected Sanger sequencing results. The methods detected even minor plasmid populations with sequence variations.

Moreover, when the plasmid sequence contained a discrete number of polyA's in the tail, the LC-MS analysis revealed a distribution that included tails both longer and shorter than the encoded tail lengths.

### Definitions

So that the invention can be more readily understood, some terms here defined, Additional definitions may be set forth throughout the specification.

“3” when used in a nucleotide position refers to a region or position in a polynucleotide or oligonucleotide 3′ (i.e., downstream) from another region or position in the same polynucleotide or oligonucleotide. The terms “3′ end” and “3′ terminus”, as used herein in reference to a nucleic acid molecule, refer to the end of the nucleic acid which contains a free hydroxyl group attached to the 3′ carbon of the terminal pentose sugar.

“5′” when used in a nucleotide position refers to a region or position in a polynucleotide or oligonucleotide 5′ (i.e., upstream) from another region or position in the same polynucleotide or oligonucleotide. The term “5′ end” and “5′ terminus”, as used herein in reference to a nucleic acid molecule, refers to the end of the nucleic acid molecule which contains a free hydroxyl or phosphate group attached to the 5′ carbon of the terminal pentose sugar. In some embodiments, oligonucleotide primers comprise tracts of poly-adenosine at their 5′ termini.

“5-methylcytidine” (⁵mC) is s a modified nucleoside derived from 5-methylcytosine. 5-Methylcytosine is a methylated form of the DNA base cytosine that may be involved in the regulation of gene transcription. See, WO WO2013/052523 (Moderna Therapeutics).

“About” means, approximately the value stated. The term “about” “reflects the inherent uncertainty in any scientific measurement—i.e., repeated measurements of the same property will not yield exactly the same result due to the limitations of accuracy and precision associated with measurement and testing techniques.

“Affinity”, as used in the art, is a measure of the tightness with which a particular ligand binds to (e.g., associates non-covalently with) and/or the rate or frequency with which it dissociates from, its partner. The skilled artisan will know that several methods have been and can be used to determine affinity. Affinity is a measure of specific binding.

“Cap0” (SEQ ID NO.: 1) is a m7GpppG cap. 5′ terminal caps are commercially available, e.g., from TriLink BioTechnologies, Inc., San Diego Calif. USA.

“Chromatography” is a technique for separation of mixtures. The mixture is typically dissolved in a fluid called the “mobile phase,” which carries it through a structure holding another material called the “stationary phase.” Examples include LC and HPLC.

“IVT” is the in vitro transcription of ribonucleic acid (RNA) from a deoxyribonucleic acid (DNA) template. Many IVT techniques are known in the biotechnological arts. For information, see The Basics: In Vitro Transcription (2015), available from Thermo Fisher Scientific Inc., Waltham, Mass., USA. Many kits for in vitro transcription are commercially available.

“Initiation site” is the initiation site for mRNA transcription. The T7 polymerase promoter best transcribes when the initiating nucleotide is guanosine. It is possible to force transcription to begin with adenosine, but this greatly decreases RNA yield.

“LC” is liquid chromatography, a technique used to separate a sample into its individual parts. This separation occurs based on the interactions of the sample with the mobile and stationary phases. Many LC techniques are known in the biotechnological arts. For more information, see the Beginners Guide to UPLC (2015) and the HPLC Primer (2015), both available from Waters Corporation, Milford, Mass., USA.

“Linearization site” or “linearization sequence”. Linearization sequences could include recognition sites for restriction endonucleases (e.g. DraI, BspQI, SapI, BbsI, etc.), or ribozyme sequences (e.g. hammerhead, hairpin, hepatitis delta virus, Varkud satellite ribozymes etc.), or T7 polymerase termination sequences. The linearization site consists of a unique restriction enzyme site that, when cut, leaves a precise end for transcription to run off. Enzymes that cut outside of their recognition sites are most useful for linearization.

“Modified” means a changed state or structure of a molecule. A “modified” mRNA contains ribonucleosides that encompass modifications relative to the standard guanine (G), adenine (A), cytidine (C), and uridine (U) nucleosides. The nonstandard nucleosides can be naturally occurring or non-naturally occurring. RNA can be modified in many ways including chemically, structurally, and functionally, by methods known to those of skill in the biotechnological arts. Such RNA modifications can include, e.g., modifications normally introduced post-transcriptionally to mammalian cell mRNA. Moreover, mRNA molecules can be modified by the introduction during transcription of natural and non-natural nucleosides or nucleotides, as described in U.S. Pat. No. 8,278,036 (Karikó et al.); U.S. Pat. Appl. No. 2013/0102034 (Schrum); U.S. Pat. Appl. No. 2013/0115272 (deFougerolles et al.) and U.S. Pat. Appl. No. 2013/0123481 (deFougerolles et al.). For examples of incorporation of ψ (pseudouridine) or m⁵C (5-methylcytidine) into mRNA, see, U.S. Pat. No. 8,278,036 (Karikó et al.); WO 2015/095351 (Novartis AG); Karikó K et al. Curr. Opin. Drug Disc. Devel. 10(5): 523-532 (2007); Karikó K et al. Mol. Therap. 16(11): 1833-1840 (2008) and Anderson B R et al., Nucleic Acids Res. 38(17): 5884-5892 (2010).

“MS” is mass spectrometry, an analytical chemistry technique that helps identify the amount and type of chemicals present in a sample by measuring the mass-to-charge ratio and abundance of gas-phase ions. A mass spectrum (plural spectra) is a plot of the ion signal as a function of the mass-to-charge ratio. Many MS techniques are known in the biotechnological arts. For more information, see the MS Primer (2015) available from Waters Corporation, Milford, Mass., USA. See also, Basiri et al., Bioanalysis 6(11): 1525-1542 (2014).

“mRNA” is messenger RNA, including eukaryotic messenger RNA. Eukaryotic mRNA can begin at the 5′ end with an mRNA cap that is enzymatically synthesized after the mRNA has been transcribed by an RNA polymerase in vitro. The mRNA cap facilitates translation initiation while avoiding recognition of the mRNA as foreign and protects the mRNA from 5′ exonuclease mediated degradation.

“Nucleoside” or “nucleobase” refer to a base (adenine (A), guanine (G), cytosine (C), uracil (U), thymine (T) and analogs thereof) linked to a carbohydrate, for example D-ribose (in RNA) or 2′-deoxy-D-ribose On DNA), through an N-glycosidic bond between the anomeric carbon of the carbohydrate and the nucleobase. When the nucleobase is purine (e.g., A or G), the ribose sugar is generally attached to the N9-position of the heterocyclic ring of the purine. When the nucleobase is pyrimidine (e.g., C, T or U), the sugar is usually attached to the N1-position of the heterocyclic ring. The carbohydrate may be substituted or unsubstituted. Substituted ribose sugars include, but are not limited to, those in which one or more of the carbon atoms, for example the 2′-carbon atom, is substituted with one or more of the same or different —Cl, —F, —R, —OR, —NR₂ or halogen groups, where each R is independently H, C₁-C₆ alkyl or C₅-C₁₄ aryl, Ribose examples include ribose, 2′-deoxyribose, 2′,3′-dideoxyribose, 2′-haloribose, 2′-fluororibose, 2′-chlororibose, and 2′-alkylribose, e.g., 2′-O-methyl, 4′-alpha-anomeric nucleotides, 2*-4*- and 3*-4*-linked and other “locked” or “LNA,” bicyclic sugar modifications. See, WO 98/22489 (Takeshi Imanishi), WO 98/39352 (Exiqon A/S, Santaris Pharma A/S); and WO 99/14226 (Exiqon A/S).

“Nucleotide” is a nucleoside in a phosphorylated form (a phosphate ester of a nucleoside), as a monomer unit or within a polynucleotide polymer. A “nucleotide 5 ‘-triphosphate” is a nucleotide with a triphosphate ester group at the 5’ position, sometimes denoted as “NTP”, or “dNTP” and “ddNTP” to particularly point out the structural features of the ribose sugar. The triphosphate ester group may include sulfur substitutions for the various oxygen moieties, e.g., α-thio-nucleotide 5′-triphosphates. Nucleotides can exist in the mono-, di-, or tri-phosphorylated forms. The carbon atoms of the ribose present in nucleotides are designated with a prime character (′) to distinguish them from the backbone numbering in the bases. For a review of polynucleotide and nucleic add chemistry see Shabarova & Bogdanov, Advanced Organic Chemistry of Nucleic Acids (VCH, New York, 1994).

“Nucleic acid”, “nucleic acid molecule”, “polynucleotide” and “oligonucleotide” refer interchangeably to polymers of nucleotide monomers or analogs thereof, such as deoxyribonucleic acid (DNA) and ribonucleic acid (RNA) and combinations thereof. The nucleotides may be genomic, synthetic or semi-synthetic in origin. Unless otherwise stated, the terms encompass nucleic acid-like structures with synthetic backbones, as well as amplification products. The length of these polymers (i.e., the number of nucleotides it contains) can vary widely, often depending on their intended function or use. Polynucleotides can be linear, branched linear, or circular molecules. Polynucleotides also have associated counter ions, such as H⁺, NH₄⁺, trialkylammonium, Mg⁺, Na⁺ and the like. A polynucleotide may be composed entirely of deoxyribonucleotides, entirely of ribonucleotides, or chimeric mixtures thereof. Polynucleotides may be composed of internucleotide nucleobase and sugar analogs. Throughout the specification, whenever an oligonucleotide is represented by a sequence of letters (chosen, for example, from the four base letters: A (adenosine), C (cytidine), G (guanosine), and T (thymidine), the nucleotides are presented in the 5′ to 3′ order from the left to the right. A “polynucleotide sequence” refers to the sequence of nucleotide monomers along the polymer. Unless denoted otherwise, whenever a polynucleotide sequence is represented, the nucleotides are in 5′ to 3′ orientation from left to right. Nucleic acids, polynucleotides and oligonucleotides may be comprised of standard nucleotide bases or substituted with nucleotide isoform analogs, including, but not limited to iso-C and iso-G bases, which may hybridize more or less permissibly than standard bases, and which will preferentially hybridize with complementary isoform analog bases. Many such isoform bases are described, e.g., by Benner et al., Cold Spring Herb. Symp. Quant. Biol. 52: 53-63 (1987). Analogs of naturally occurring nucleotide monomers include, for example, 7-deazaadenine, 7-deazaguanine, 7-deaza-8-azaguanine, 7-deaza-8-azaadenine, 7-methylguanine, inosine, nebularine, nitropyrrole, nitroindole, 2-aminopurine, 2-amino-6-chloropurine, 2,6-diaminopurine, hypoxanthine, pseudouridine (ψ), pseudocytosine, pseudoisocytosine, 5-propynylcytosine, isocytosine, isoguanine), 7-deazaguanine, 2-azapurine, 2-thiopyrimidine, 6-thioguanine, 4-thiothymine, 4-thiouracil, O-6-methylguanine, N6-methyladenine, O-4-methylthymine, 5,6-dihydrothymine, 5,6-dihydrouracil, 4-methylindole, pyrazolo[3,4-D]pyrimidines, “PPG” (U.S. Pat. Nos. 6,143.877 (Meyer)) and ethenoadenine (Fasrnan, in Practical Handbook of Biochemistry and Molecular Biology, pp. 385-394 (CRC Press, Boca Raton Fla. USA, 1989)). See also, the nucleosides described in U.S. Pat. No. 8,278,036 (Karikó et al.); WO 2015/095351 (Novartis AG); Karikó K et al. Curr. Opin. Drug Disc. Devel. 10(5): 523-532 (2007); Karikó K et al. Mol. Therap. 16(11): 1833-1840 (2008) and Anderson B R et al., Nucleic Acids Res. 38(17): 5884-5892 (2010).

“Oligo dT” is a stretch of deoxy-thymidine nucleotides, often used to hybridize to and purify molecules containing polyA, such as mRNA. Oligo dT and oligi Dt magnetic beads are commercially available.

“PolyA tail”. The polyA tail is important for binding of translational factors and for stability. The polyA tail will be at the 3′end of mRNA can range in length.

“Polynucleotide variant” refers to molecules that differ in their nucleotide sequence from a native or reference sequence, which can possess substitutions, deletions, or insertions at certain positions within the amino acid sequence, as shown in WO 2015/006747 A2.

“Primers” are short nucleic acid sequences. Polymerase chain reaction (PCR) primers are typically oligonucleotides of short length (e.g., 8-30 nucleotides) that are used in polymerase chain reactions. PCR primers and hybridization probes can readily be developed and produced by those of skill in the art, using sequence information from the target sequence. See, Green & Sambrook, Molecular Cloning: A Laboratory Manual, Fourth Edition (Cold Spring Harbor Press, Plainview, N.Y., 2012).

A “probe” as used herein is an oligonucleotide probe, a nucleic acid molecule which typically ranges in size from about 50-100 nucleotides to several hundred nucleotides to several thousand nucleotides in length, in whole number increments. A probe can be any suitable length for use in the method of the invention described herein. Such a molecule is typically used to identify a specific nucleic acid sequence in a sample by hybridizing to the specific nucleic acid sequence under stringent hybridization conditions. Hybridization conditions are known in the biotechnological arts. See, e.g., Green & Sambrook, Molecular Cloning: A Laboratory Manual, Fourth Edition (Cold Spring Harbor Press, Plainview, N.Y., 2012).

“Pseudouridine” (ψ) is an isomer of the nucleoside uridine in which the uracil is attached via a carbon-carbon instead of a nitrogen-carbon glycosidic bond. See, WO WO2013/052523 (Moderna Therapeutics).

“RNA” is ribonucleic acid, a ribonuceloside polymer. Each nucleotide in an RNA molecule contains a ribose sugar, with carbons numbered 1′ through 5′. A base is attached to the 1′ position. In general, the bases are adenine (A), cytosine (C), guanine (G), or uracil (U), although many modifications are known to those of skill in the art. For example, as described herein, an RNA may contain one or more pseudouracil (ψ) base, such that the pseudouridine nucleotides are substituted for uridine nucleotides. Many other RNA modifications are known to those of skill in the art, as described herein. Procedures for isolating and producing RNA are known to the skilled artisan, such as a laboratory scientist. See, Green & Sambrook, Molecular Cloning: A Laboratory Manual, Fourth Edition (Cold Spring Harbor Press, Plainview N.Y., 2012).

“Restriction site for linearization of plasmid DNA template” is a site in the plasmid DNA template that can be cut to generate a linearized DNA, for use in in vitro transcription.

A “substitution” is a mutation that exchanges one base for another (i.e., a change in a single “chemical letter” such as switching an A to a G). Such a substitution could (a) change a codon to one that encodes a different amino acid and cause a small change in the protein produced; (b) change a codon to one that encodes the same amino acid and causes no change in the protein produced (“silent mutations”); or (c) change an amino-acid-coding codon to a single “stop” codon and cause an incomplete protein.

A “surface coated substrate” is a substrate that is coated with a reagent that binds to a nonradiolabeled tagged probe. In one embodiment, the substrate of the surface coated substrate is a magnetic bead. In one embodiment, the substrate of the surface coated substrate is a polymeric bead. In one embodiment, the substrate of the surface coated substrate is a well-plate.

“SP6 polymerase” is a DNA-dependent RNA polymerase from the SP6 bacteriophage that catalyzes the formation of RNA in the 5′→3′.

“T1” is ribonyclease (RNAse) T1 (EC 3.1.27.3) is a fungal endonuclease that cleaves single-stranded RNA after guanine residues, i.e., on their 3′ end. The most commonly studied form of this enzyme is the version found in the mold Aspergillus oryzae. RNase T1 is often used to digest denatured RNA prior to sequencing. RNase T1 is commercially available, e.g., Life Technologies # AM2280 1000units/μL.

“T3 polymerase” is a DNA-dependent RNA polymerase from the T3 bacteriophage that catalyzes the formation of RNA in the 5′→3′.

“T7 polymerase” is a DNA-dependent RNA polymerase from the T7 bacteriophage that catalyzes the formation of RNA in the 5′→3′.

“T7 polymerase promoter upstream enhancer sequence”, SEQ ID NO.: 2, is an enhancer sequence upstream from the T7 polymerase promoter, which helps to increase the yield of RNA in an IVT reaction.

“T7 polymerase promoter”, SEQ ID NO. 3 is a nucleotide sequence for a T7 polymerase to begin transcription. Transcription initiates on the first nucleotide following the promoter sequence (typically guanosine).

The term “target” refers to a molecule of interest “Target RNA” is an RNA of interest, which can be analyzed by the method of the invention.

“Transcription initiation nucleotide” is the first nucleotide from which transcription begins. A transcription initiation nucleotide could be A, T, C or G, depending on promoter and RNA-polymerase chosen for specific transcript.

“Transcription” is the first step of gene expression, in which a particular segment of DNA is copied into RNA by the enzyme RNA polymerase.

“Upstream” refers to the 5′ to 3′ direction in which RNA transcription takes place, so downstream is toward the 5′ end of an RNA molecule.

### Methods

The methods described herein allow for the determination of the polyA tail species present in a composition of RNA, e.g., mRNA, without the need for radiolabeling, by using mass spectrometry, e.g., LC-MS or MALDI-MS, to detect the differences in mass and retention time of the polyA tail species within the composition. Applicants discovered that mass spectrometry provides accurate and high-resolution identification of polyA tail lengths in RNA compositions. The methods described herein, are useful for evaluating or processing a RNA composition, e.g., an mRNA composition, to determine whether to accept or reject a batch of RNA, or to guide or control a step in the production of a RNA composition, e.g., an mRNA composition.

Accordingly, in one aspect, the disclosure features a method of analyzing the quality of an RNA composition, e.g., an mRNA composition, e.g., by evaluating or processing the RNA composition, e.g., mRNA composition, for and/or based upon the polyA chain lengths of the various polyA tails of the RNA, e.g., mRNA, in the composition using mass spectrometry, e.g., LC-MS or MALDI-MS. The analysis of the RNA composition, e.g., mRNA composition, by mass spectrometry can be used to evaluate processes, intermediates and final products in the production of RNA compositions, e.g., mRNA compositions. The presence, distribution or amount of polyA tail length species can be used in these evaluations.

In one embodiment, the method comprises providing an evaluation of a parameter, e.g., the relative distribution of polyA chain lengths or amount of a polyA chain length in the RNA composition, to provide a test value and, optionally, providing a determination of whether the parameter meets a preselected criteria, e.g., is present in an amount or has a profile of a reference value, thereby evaluating or processing the RNA composition. By way of example, LC-MS analysis of a sample of an RNA composition can resolve mixtures of polyA lengths of the RNA in the composition and provide a profile of polyA chain lengths within the composition.

In one embodiment, the test value, or an indication of whether the preselected relationship is met, can be memorialized, e.g., in a computer readable record.

In one embodiment, a decision or step is taken, e.g., the RNA composition is classified, selected, accepted or discarded, released or withheld, processed into a drug product, shipped, moved to a different location, formulated, labeled, packaged, released into commerce, or sold or offered for sale, depending on whether the preselected relationship is met. E.g., based on the result of the determination, or upon comparison to a reference standard, the RNA composition, e.g., mRNA composition, from which the sample is taken can be processed, e.g., as just described.

In one embodiment, the method comprises providing isolated polyA tails cleaved from a sample of the RNA composition and determining a parameter, e.g., the profile and/or the lengths, of the polyA tails from the sample using mass spectrometry, e.g., LC-MS or MALDI-MS, to thereby determine the quality of the RNA composition.

In one embodiment, the polyA tail is cleaved from the RNA in the sample using an enzyme or combination of enzymes that do not cleave adenosine such as, e.g., ribonuclease T1, RNAse CL3 (cusativin) and RNase A. In one embodiment, the method further comprises cleaving the polyA tails from the RNA, e.g., mRNA, in the sample, e.g., using an enzyme or combination of enzymes that do not cleave adenosine such as, e.g., ribonuclease T1, RNAse CL3 (cusativin) and RNase A. In one embodiment, the method further comprises isolating the cleaved polyA tails from the RNA in the sample by hybridization to surface coated substrate conjugated with polynucleotides. The method described herein for polyA tail analysis advantageously provides single nucleotide resolution of the cleaved polyA tails.

In one embodiment, the method comprises isolating the cleaved polyA tails from the RNA in the sample and the surface coated substrates comprise magnetic beads. In one embodiment, the polynucleotide conjugated to the surface coated substrates is oligo dT (a stretch of deoxy-thymidine nucleotides). In one embodiment, the surface coated substrates are magnetic beads and the polynucleotide conjugated to the magnetic beads is oligo dT.

In one embodiment, the RNA is made by in vitro transcription (IVT), e.g., an in vitro transcription method described herein. In one embodiment, the in vitro transcribed RNA is an mRNA. In one embodiment, the in vitro synthesized RNA comprises modified nucleotides selected from, e.g.: ψ (pseudouridine); m⁵C (5-methylcytidine); m⁵U (5-methyluridine); m⁶A (N⁶-methyladenosine); s²U (2-thiouridine); Um (2′-O-methyl-U; 2′-O-methyluridine); m¹A (1-methyladenosine); m²A (2-methyladenosine); Am (2′-O-methyladenosine); ms² m⁶A (2-methylthio-N⁶-methyladenosine); i⁶A (N⁶-isopentenyladenosine); ms²i⁶A (2-methylthio-N⁶isopentenyladenosine); io⁶A (N⁶-(cis-hydroxyisopentenyl)adenosine); ms²i⁶A (2-methylthio-N⁶-(cis-hydroxyisopentenyl)adenosine); g⁶A (N⁶-glycinylcarbamoyladenosine); t⁶A (N⁶-threonylcarbamoyladenosine); ms²t⁶A (2-methylthio-N⁶-threonyl carbamoyladenosine); m⁶t⁶A (N⁶-methyl-N⁶-threonylcarbamoyladenosine); hn⁶A (N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m1Im (1,2′-O-dimethylinosine); m³C (3-methylcytidine); Cm (2′-O-methylcytidine); s²C (2-thiocytidine); ac⁴C(N⁴-acetylcytidine); f⁵C (5-formylcytidine); m⁵ Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²₂Gm (N²,N²,2′-O-trimethylguanosine); Gr(p) (2′-O-ribosylguanosine (phosphate)); yW (wybutosine); o₂yW (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ, (7-aminomethyl-7-deazaguanosine); G+(archaeosine); D (dihydrouridine); m⁵Um (5,2′-O-dimethyluridine); s⁴U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s²U (5-methoxycarbonylmethyl-2-thiouridine); nm⁵s²U (5-aminomethyl-2-thiouridine); mnm⁵U (5-methylaminomethyluridine); mnm⁵s²U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxmethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C(N⁴-methylcytidine); m⁴ Cm (N⁴,2′-O-dimethylcytidine); hm⁵C (5-hydroxymethylcytidine); m³U (3-methyluridine); cm⁵U (5-carboxymethyluridine); m⁶Am (N⁶,2′-O-dimethyladenosine); m⁶ ₂Am (N⁶,N⁶,O-2′-trimethyladenosine); m²′⁷G (N²,7-dimethylguanosine); m²′²′⁷G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); andac⁶A (N⁶-acetyladenosine), and combinations thereof.

In one embodiment, the methods described herein are completed within 5 hours, with most of that time spent incubating for enzymatic cleavage.

In one embodiment, the polyA tails present in the sample can be from about 10 A's to about 300 A's (e.g., 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, or 300 A's) in length or longer. In one embodiment, the polyA tails present in the sample can be from about 20 A's to about 200 A's in length. In one embodiment, the polyA tails present in the sample can be from about 20 A's to about 120 A's in length. In one embodiment, the polyA tails present in the sample can be from about 30 A's to about 120 A's in length. In one embodiment, the polyA tails present in the sample can be from about 40 A's to about 120 A's in length. In one embodiment, the polyA tails present in the sample can be from about 50 A's to about 120 A's in length. In one embodiment, the polyA tails present in the sample can be from about 60 A's to about 120 A's in length. In one embodiment, the polyA tails present in the sample can be from about 20 A's to about 190 A's in length. In one embodiment, the polyA tails present in the sample can be from about 20 A's to about 180 A's in length. In one embodiment, the polyA tails present in the sample can be from about 20 A's to about 170 A's in length. In one embodiment, the polyA tails present in the sample can be from about 20 A's to about 160 A's in length. In one embodiment, the polyA tails present in the sample can be from about 20 A's to about 150 A's in length. In one embodiment, the polyA tails present in the sample can be from about 20 A's to about 140 A's in length. In one embodiment, the polyA tails present in the sample can be from about 20 A's to about 130 A's in length.

The methods described herein are useful for analyzing or processing a RNA composition, e.g., to determine whether to accept or reject a batch of a RNA composition or to guide or control a step in the production of a RNA composition.

In one embodiment, the method further comprises a step of further processing the RNA composition. The further processing can be, e.g., one or more of selecting, accepting, processing into a drug product, shipping, formulating, labeling, packaging, or selling the RNA composition. In one embodiment, the further processing comprises processing the RNA composition into a drug product. In another embodiment, the further processing comprises formulating the RNA.

In one embodiment, the RNA composition comprises an RNA drug substance. In one embodiment, the RNA composition is an RNA drug product.

In one embodiment, the evaluation comprises determining if the batch of the RNA composition meets a predetermined reference value, and optionally memorializing the determination, e.g., in a computer readable record.

In one embodiment, the reference value is a value determined from a sample of a commercially available RNA composition. In one embodiment, the reference value is a value determined from a previous batch of the RNA composition. In one embodiment, the reference value is or comprises a production standard imposed by a regulatory agency, e.g., a release standard.

In one embodiment, the method further comprises altering a step in the production of an RNA composition based upon the determination, e.g., modifying, e.g., increasing or decreasing, the amount of ATPase used the production process of the RNA composition and/or modifying, e.g., increasing or decreasing, the amount of ATP used in the production process.

In another aspect, the disclosure features a method of making an RNA composition, e.g., an mRNA composition, comprising providing an RNA sample from a RNA composition, providing an evaluation of a parameter, e.g., the relative distribution of polyA chain lengths or amount of a polyA chain length in the RNA sample, by mass spectrometry, e.g., LC-MS or MALDI-MS, to provide a test value; providing a determination of whether the parameter meets a preselected criteria, e.g., is present in an amount or has a profile of a reference value, and further processing the RNA composition based upon the determination. The further processing can be, e.g., classifying, selecting, accepting or discarding, releasing or withholding, processing into a drug product, shipping, moving to a different location, formulating, labeling, packaging, releasing into commerce, or selling or offering for sale, based upon whether the preselected relationship is met. E.g., based on the result of the determination, or upon comparison to a reference standard, the RNA composition from which the sample is taken can be processed, e.g., as just described.

In one embodiment, the preselected criteria is met, e.g., the RNA sample has an amount of a polyA chain length or has a profile of polyA chain length distribution of a reference value, and the RNA composition is processed into drug product, formulated, labeled, packaged, released into commerce based upon the determination. In one embodiment, the preselected criteria is met, e.g., the RNA sample has an amount of a polyA chain length or has a profile of polyA chain length distribution of a reference value and the production method used to make the RNA composition is used to make additional batches of the RNA composition. In one embodiment, when the preselected criteria is met, it is predictive of or ensures that a batch of RNA composition will meet a release specification.

In one embodiment, the preselected criteria is not met, e.g., the RNA sample does not have an amount of a polyA chain length or does not have a profile of polyA chain length distribution of a reference value, and the RNA composition is discarded or withheld. In one embodiment, the preselected criteria is not met, e.g., the RNA sample does not have an amount of a polyA chain length or does not have a profile of polyA chain length distribution of a reference value, and the production method used to make the RNA composition is modified. For example, the amount of ATPase used the production process of the RNA composition is modified, e.g., increased or decreased, in subsequent batches of the RNA composition and/or the amount of ATP used in the production process is modified, e.g., increased or decreased, in subsequent batches of the RNA composition.

In one embodiment, the method comprises evaluating the RNA sample, e.g., by the methods described herein.

In any method described herein, in some embodiments, the method includes a step of cleaving the polyA tails from the mRNA in the sample using an enzyme or combination of enzymes that do not cleave adenosine. In some embodiments, such cleavage takes about 1 hour, 2 hours, or 3 hours.

### Reference Values and Standards

In some embodiments, the methods described herein include providing a comparison of the test value determined with a reference value or values, to thereby evaluate the RNA sample. In preferred embodiments, the comparison includes determining if the test value has a preselected relationship with the reference value, e.g., determining if it meets the reference value. The value need not be a numerical value but, e.g., can be merely an indication of whether the subject entity is present.

In one embodiment, the method includes determining if a test value is equal to or greater than a reference value, if it is less than or equal to a reference value, or if it falls within a range (either inclusive or exclusive of one or both endpoints). By way of example, the amount of the relative distribution of polyA chain lengths in the RNA sample can be determined and, optionally shown to fall within a preselected range, e.g., a range which corresponds to a range of the reference value.

A reference value, by way of example, can be a value determined from a reference sample (e.g., a commercially available sample or a sample from previous production). The reference value can be numerical or non-numerical. For example, it can be a qualitative value, e.g., yes or no, or present or not present at a preselected level of detection, or graphic or pictorial. The reference value can also be values for the presence of more than one polyA chain length in an RNA sample. For example, the reference value can be a map of structures present in RNA sample when analyzed by mass spectrometry, e.g., LC-MS, e.g., an LC-MS method described herein. The reference value can also be a release standard (a release standard is a standard which should be met to allow commercial sale of a product) or production standard, e.g., a standard which is imposed, e.g., by a party, e.g., the FDA, on an RNA composition.

The reference value can be derived from any of a number of sources. The reference value can be one which was set or provided by (either solely or in conjunction with another party, e.g., a regulatory agency, e.g., the FDA), the manufacturer of the drug or practitioner of a process to make the drug. The reference value can be one which was set or provided by (either solely or in conjunction with another party, e.g., a regulatory agency, e.g., the FDA), a party other than the party manufacturing a drug and practicing a method disclosed herein, e.g., another party which manufactures the drug or practices a process to make the drug. The reference value can be one which was set or provided by (either solely or in conjunction with another party) a regulatory agency, e.g., the FDA, to the manufacturer of the drug or practitioner of the process to make the drug, or to another party licensed to market the drug. For example, the reference standard can be a production, release, or product standard required by the FDA. In one embodiment, a reference value is a value required of a pioneer drug (e.g., a drug marketed under an approved NDA) or a generic drug (e.g., a drug marketed or submitted for approval under an ANDA).

The reference value can be a statistical function, e.g., an average, of a number of values.

In some embodiments, the reference value refers to a distribution where at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 95%, 97%, 98%, or 99%) of produced polyAs are within the expected length range.

In some embodiments, the reference value refers to a distribution where at least 75% of produced polyAs are within the expected length range.

In some embodiments, the reference value refers to a distribution where at least 76% of produced polyAs are within the expected length range.

In some embodiments, the reference value refers to a distribution where at least 77% of produced polyAs are within the expected length range.

In some embodiments, the reference value refers to a distribution where at least 78% of produced polyAs are within the expected length range.

In some embodiments, the reference value refers to a distribution where at least 79% of produced polyAs are within the expected length range.

In some embodiments, the reference value refers to a distribution where at least 80% of produced polyAs are within the expected length range.

In some embodiments, the reference value refers to a distribution where at least 81% of produced polyAs are within the expected length range.

In some embodiments, the reference value refers to a distribution where at least 82% of produced polyAs are within the expected length range.

In some embodiments, the reference value refers to a distribution where at least 83% of produced polyAs are within the expected length range.

In some embodiments, the reference value refers to a distribution where at least 84% of produced polyAs are within the expected length range.

In some embodiments, the reference value refers to a distribution where at least 85% of produced polyAs are within the expected length range.

In some embodiments, the reference value refers to a distribution where at least 86% of produced polyAs are within the expected length range.

In some embodiments, the reference value refers to a distribution where at least 87% of produced polyAs are within the expected length range.

In some embodiments, the reference value refers to a distribution where at least 88% of produced polyAs are within the expected length range.

In some embodiments, the reference value refers to a distribution where at least 89% of produced polyAs are within the expected length range.

In some embodiments, the reference value refers to a distribution where at least 90% of produced polyAs are within the expected length range.

In some embodiments, the reference value refers to a distribution where at least 91% of produced polyAs are within the expected length range.

In some embodiments, the reference value refers to a distribution where at least 92% of produced polyAs are within the expected length range.

In some embodiments, the reference value refers to a distribution where at least 93% of produced polyAs are within the expected length range.

In some embodiments, the reference value refers to a distribution where at least 94% of produced polyAs are within the expected length range.

In some embodiments, the reference value refers to a distribution where at least 95% of produced polyAs are within the expected length range.

In some embodiments, the reference value refers to a distribution where at least 96% of produced polyAs are within the expected length range.

In some embodiments, the reference value refers to a distribution where at least 97% of produced polyAs are within the expected length range.

In some embodiments, the reference value refers to a distribution where at least 98% of produced polyAs are within the expected length range.

In some embodiments, the reference value refers to a distribution where at least 99% of produced polyAs are within the expected length range.

In some embodiments, the expected length range is ±20nt of the expected length. For example, if the expected polyA length is 120nt (SEQ ID NO: 12) (based on the corresponding DNA template), the expected length range is between 100nt and 140nt.

In some embodiments, the expected length range is ±15nt of the expected length. For example, if the expected polyA length is 120nt (SEQ ID NO: 12) (based on the corresponding DNA template), the expected length range is between 105nt and 135nt.

In some embodiments, the expected length range is ±10nt of the expected length. For example, if the expected polyA length is 120nt (SEQ ID NO: 12) (based on the corresponding DNA template), the expected length range is between 110nt and 130nt.

In some embodiments, the expected length range is ±5nt of the expected length. For example, if the expected polyA length is 120nt (SEQ ID NO: 12) (based on the corresponding DNA template), the expected length range is between 115nt and 125nt.

In some embodiments, the expected length range is +5nt of expected length. For example, if the expected polyA length is 120nt (SEQ ID NO: 12) (based on the corresponding DNA template), the expected length range is between 120nt and 125nt.

In some embodiments, the expected length range is +6nt of the expected length. For example, if the expected polyA length is 120nt (SEQ ID NO: 12) (based on the corresponding DNA template), the expected length range is between 120nt and 126nt.

In some embodiments, the expected length range is +7nt of the expected length. For example, if the expected polyA length is 120nt (SEQ ID NO: 12) (based on the corresponding DNA template), the expected length range is between 120nt and 127nt.

In some embodiments, the expected length range is +8nt of the expected length. For example, if the expected polyA length is 120nt (SEQ ID NO: 12) (based on the corresponding DNA template), the expected length range is between 120nt and 128nt.

In some embodiments, the expected length range is +9nt of the expected length. For example, if the expected polyA length is 120nt (SEQ ID NO: 12) (based on the corresponding DNA template), the expected length range is between 120nt and 129nt.

In some embodiments, the expected length range is +10nt of the expected length. For example, if the expected polyA length is 120nt (SEQ ID NO: 12) (based on the corresponding DNA template), the expected length range is between 120nt and 130nt.

In some embodiments, the expected length range is +11nt of the expected length. For example, if the expected polyA length is 120nt (SEQ ID NO: 12) (based on the corresponding DNA template), the expected length range is between 120nt and 131nt.

In some embodiments, the expected length range is +12nt of the expected length. For example, if the expected polyA length is 120nt (SEQ ID NO: 12) (based on the corresponding DNA template), the expected length range is between 120nt and 132nt.

In some embodiments, the expected length range is +13nt of the expected length. For example, if the expected polyA length is 120nt (SEQ ID NO: 12) (based on the corresponding DNA template), the expected length range is between 120nt and 133nt.

In some embodiments, the expected length range is +14nt of the expected length. For example, if the expected polyA length is 120nt (SEQ ID NO: 12) (based on the corresponding DNA template), the expected length range is between 120nt and 134nt.

In some embodiments, the expected length range is +15nt of the expected length. For example, if the expected polyA length is 120nt (SEQ ID NO: 12) (based on the corresponding DNA template), the expected length range is between 120nt and 135nt.

In some embodiments, the expected length range is +16nt of the expected length. For example, if the expected polyA length is 120nt (SEQ ID NO: 12) (based on the corresponding DNA template), the expected length range is between 120nt and 136nt.

In some embodiments, the expected length range is +17nt of the expected length. For example, if the expected polyA length is 120nt (SEQ ID NO: 12) (based on the corresponding DNA template), the expected length range is between 120nt and 137nt.

In some embodiments, the expected length range is +18nt of the expected length. For example, if the expected polyA length is 120nt (SEQ ID NO: 12) (based on the corresponding DNA template), the expected length range is between 120nt and 138nt.

In some embodiments, the expected length range is +19nt of the expected length. For example, if the expected polyA length is 120nt (SEQ ID NO: 12) (based on the corresponding DNA template), the expected length range is between 120nt and 139nt.

In some embodiments, the expected length range is +20nt of the expected length. For example, if the expected polyA length is 120nt (SEQ ID NO: 12) (based on the corresponding DNA template), the expected length range is between 120nt and 140nt.

In some embodiments, the expected length range is within the range of −Xnt to +Ynt of the expected length, where X is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30; and Y is 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30.

In some embodiments, the expected length or the expected polyA length is the length of the polyA tail based on the number of nucleotides (e.g., adenosines) in corresponding DNA template.

Mass Spectrometry

The evaluating step can include mass spectral and/or tandem mass spectrometry (MS/MS) techniques. In this technique, parent nucleotide ions are fragmented into smaller ions which are selected and further fragmented to yield information relating to the nature of the polyA nucleotide mixture. To characterize a type of polyA mixture by mass spectrometry, a type of nucleotide or a particular segment of a type of nucleotide can be given positive and negative charges, or ionized, and volatilized in a mass spectrometer. The ionized, volatilized nucleotide molecules or segment thereof can then analyzed by the mass spectrometer, which produces a mass spectrum of the nucleotide molecule or segment.

A mass spectrometer determines the weight and/or retention of nucleotide molecules and segments of nucleotide molecules, when a nucleotide molecule or segment is analyzed, the information provided by mass spectrometry can be of used, e.g., to determine the lengths of various polyA nucleotide segments, present in an RNA sample. Methods such as matrix assisted laser desorption ionization (MALDI), nanospray GC/MS, LC/MS, LC MS/MS are all encompassed within the meaning of mass spectrometry.

Mass spectrometry is a direct measurement technique that does not require labels. Mass spectrometry can distinguish between single nucleotides by their differences in mass.

### REFERENCES

The following references may be useful in the practice of the instant invention,
PCT patent publication WO 20171098468 A1 (Novartis AG).
Aitken & Lorsch, A mechanistic overview of translation initiation in eukaryotes. Nature Struct. Mol. Biol. 19: 568-576 (2012).
Beverly, et al., Label-free analysis of mRNA capping efficiency using RNase H probes and LC-MS. Anal. Bioanal. Chem. 408: 5021 ((2016))
Gilar, Analysis and purification of synthetic oligonucleotides by reversed-phase high-performance liquid chromatography with photodiode array and mass spectrometry detection. Anal. Biochem. 298: 196-206 (2001).
Harrison et al., PAT-seq: a method to study the integration of 3′-UTR dynamics with gene expression in the eukaryotic transcriptome. RNA 21: 1502-1510 (2015).
Moqtaderi et al., Secondary structures involving the poly(A) tail and other 3′ sequences are major determinants of mRNA isoform stability in yeast. Microb. Cell 1: 137-139 (2014).

### EXAMPLES

### Example 1

Presented herein are some results from using the LC-MS methods described herein for the characterization of 2100nt IVT synthesized mRNAs, having plasmid-encoded polyA tail lengths between 27 and 117 nucleotides in length (SEQ ID NO: 13).

A sample of 2100nt IVT-synthesized mRNAs was digested with RNAse T1. The cleavage fragments containing polyA stretches were then isolated using oligo dT coated magnetic beads. After washing to remove unbound cleavage products and reaction buffer, the dT bound species were eluted off the beads and then injected into the LC-MS machine for analysis. After analysis, the collected electrospray mass spectra are processed and identified by mass using the known sequence of the mRNA cleavage products.

The measured distribution of tail lengths closely matched the sequence populations observed in Sanger sequencing.

DNA Template preparation:

DNA plasmids were generated using conventional cloning methods. Linearized plasmid DNA templates were analyzed by gel electrophoresis and Sanger nucleotide sequencing prior to mRNA preparation.

T7, SP3, or T3 RNA polymerase promoter sequences were encoded within the plasmids to generate mRNA using different RNA polymerase forms.

For some plasmids, unique restriction enzyme sites were added to the DNA plasmids to allow for the production of polyA encoded templates or tailless DNA templates. A NotI restriction enzyme site was placed upstream to the polyA tail, so that linearization with this enzyme generated DNA templates lacking the polyA tail. The BspQI, BbsI, or XhoI restriction sites were placed at the end of the polyA tail and linearization of the DNA with these enzymes generated plasmid DNA templates with polyA tail encoded.

mRNA Preparation:

mRNA was prepared using standard run-off IVT procedures and then capped (Cap 0; SEQ ID NO: 1) using the Vaccinia capping system (Part # M2080S, New England Biolabs, Ipswich, Mass., USA). Following capping, the mRNA was precipitated with LiCl and then brought up in water.

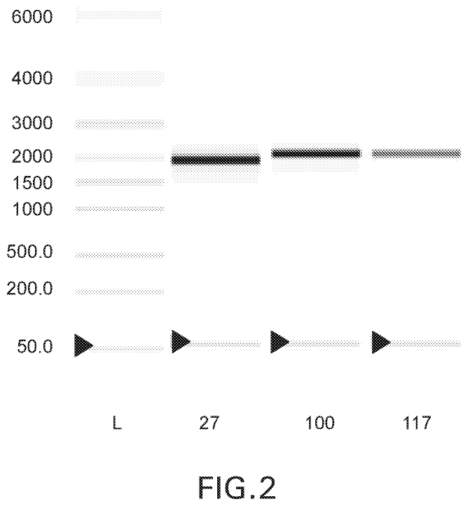

The mRNA DNA template was linearized by either an XhoI or BbsI, or BspQI, or NotI endonucleases to ensure 3′ ends of mRNA has either only stretches of adenosines (BbsI or BspQI), or extra sequence after polyA (XhoI), or no polyA (NotI). The length and integrity of the mRNA was confirmed with gel electrophoresis (BioRad Experion, Hercules, Calif., USA). See, FIG. 2.

Oligonucleotides used to validate the method were purchased from Integrated DNA Technologies (Coralville, Iowa, USA) and were brought up in DI water and used directly.

T1 Cleavage and polyA Tail Isolation Procedure:

100-150pmol of oligonucleotide or mRNA (previously heated to 95° C. and then quickly cooled) in water was added to an Eppendorf tube along with 10 μL of 10×RNAse H buffer (New England Biolabs, Ipswich, Mass., USA) and 2 μL of RNAse T1 (Life Technologies # AM2280 1000 units/μL) followed by water to make a 1× buffer (1× Buffer: 5 mM Tris pH 7.5, 0.5 mM EDTA, 1M NaCl). The sample was then vortexed briefly and kept at 37° C. for 3 hours. After 3 hours 100 μL of the mixture was added to 75 μL of oligo d) 25 magnetic beads (Dynabeads mRNA Purification Kit, #61006, Invitrogen) and isolated according to the manufacturer's protocol. The final rinse step in the protocol was changed to using 100 μL of 100 mM ammonium acetate instead of 100 μL washing buffer in order to remove any sodium that might interfere with the LC-MS analysis.

Using the magnet, the ammonium acetate wash was removed from the beads. Next 100 μL of 75% methanol (MeOH) that had been heated to 80° C. was added to the beads to release the bound polyA tails. The methanol bead mixture was heated to 80° C. on a hot plate for 1 min and placed on a magnet for 1 min. The supernatant containing the cleavage product and released polyA tail was collected and dried down to 10 μL with an evaporative centrifuge at RT for ˜45 min. Finally, the sample was resuspended in 50 μL of 100 μM EDTA/1% MeOH for LC-MS analysis.

LC-MS Analysis:

Standard conditions reported in the literature were used for the LC-MS analysis of oligonucleotides. See, Apffel et al., Anal. Chem. 69: 1320-1325 (1997); Gilar, Anal. Biochem. 298: 196-206 (2001); Huber & Oberacher, Mass Spectrom. Rev. 20: 310-343 (2001). Analysis of the cleaved 3′ mRNA fragment was conducted with an Acuity UPLC (Waters, Milford, Mass., USA) equipped with a TUV detector that was connected to a QExactive orbitrap (Thermo Scientific, Grand Island, N.Y., USA). Mobile phase A consisted of 200 mM hexafluoro isopropanol+8.15 mM triethylamine, pH 7.9, and mobile phase B was 100% MeOH See, Apffel et al., Anal. Chem. 69: 1320-1325 (1997). A Waters Acuity C18 BEH, 2.1×100 mm column heated to 75° C. with a flow rate of 300 μl/min was used for all analyses. The gradient profile for elution started at 5% B for 1 min followed by a linear ramp to 25% over 12 min. At 12 min, a one min rinse at 90% B began, followed by a return to 5% B at 13 min. UV analysis at 260 nm was conducted online prior to the MS.

All mass spectra were obtained in the negative ion mode, over a scan range of 800-2500 m/z at 35,000 resolution. Source and capillary temperatures were set to 350° C. and all spectra were analyzed using Promass Software (Novatia, Newtown, Pa., USA). See, Hail et al., American Biotechnology Laboratory 12-13 (January 2004). The deconvolution output mass range was set between 10,000 to 50,000 amu with m/z input of 900-2000 m/z, and a deconvolution peak width of 1.25 amu.

Protein Expression Assay

Luciferase expression of mRNA was assessed by monitoring fluorescence in C2C12 cells 24 h post-transfection. Cells were plated at 5000 cells/well 24 h prior to the transfection, and 25 ng of mRNA was transfected into the cells using TranslT transfection reagent (Minis, Madison, W).

Validation of the method of the invention. Sample mRNA is first digested with RNAse T1 which cleaves phosphodiester bonds at the 3′ side of guanine. Cleavage fragments containing polyA stretches are then isolated using oligo dT-coated magnetic beads. Magnetic beads functionalized with strands of polythymidine DNA (usually 25 mer in length) are commonly employed for mRNA isolation and use the poly A tail found in mRNA as a handle for capture via Watson-Crick base pairing. After washing the beads to re-move unbound cleavage products and reaction buffer, the bound poly A species are eluted off the beads and then injected into the LC-MS for analysis. After analysis, the collected electrospray mass spectra are processed and using the known sequences of the mRNA T1 cleavage products are identified by mass.

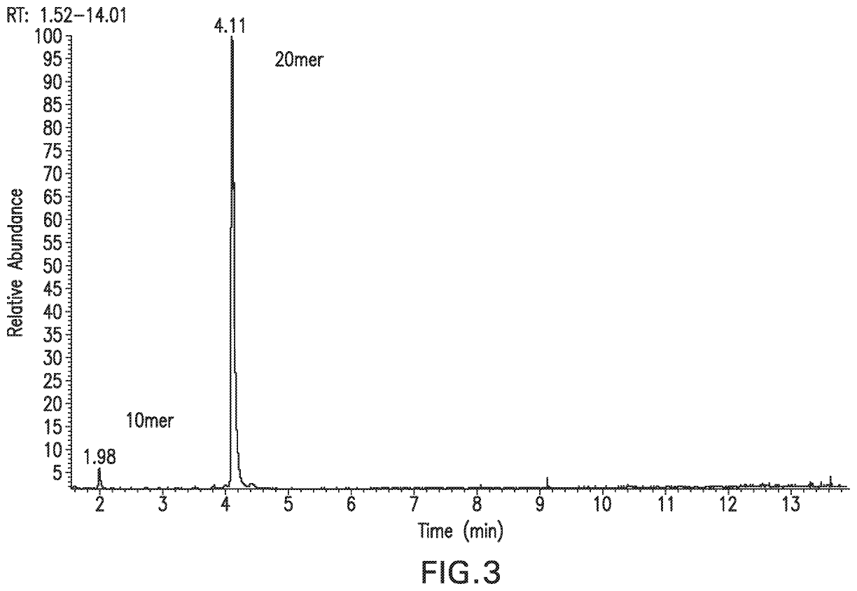

The method was validated using two synthetic RNAs, CCUGAAAAAAAAAA (SEQ ID NO.: 4) and CCUGAAAAAAAAAAAAAAAAAAAA (SEQ ID NO.: 5). These synthetic RNAs were designed to produce a 10 mer or 20 mer polyA (SEQ ID NOS 9 and 10) product following T1 digestion. Accordingly, we conducted T1 digestions of a mixture (100pmol each) of each oligo at each of 1, 3 and 24 hours, which digestions were followed by dT isolation of the polyA tails and LC-MS analysis. Our 1 hr and 3 hr incubations each produced a single fragment corresponding to the cleaved polyA 10 mer and 20 mer (SEQ ID NOS 9 and 10) products. Complete digestion of the starting oligo was achieved, as shown by the presence of a single fragment. See, FIG. 3. The 24 hr digestion time however, showed cleavage of the polyA strand itself. Accordingly, we selected the 3 hr incubation time for all subsequent analyses. The chromatographic peak of the 10 mer was substantially smaller than the 20 mer indicating that the smaller strand was captured less efficiently than the 20 mer. See, FIG. 3. We expected this less efficient capture result, considering the Tm and binding strength of the 10 mer as compared to the 20 mer.

Using the methods described herein, a 2100nt long mRNA with plasmid encoded tail lengths of 27 (SEQ ID NO: 6), 64 (SEQ ID NO: 11), 100 (SEQ ID NO: 7), 117 polyA's (SEQ ID NO: 8), along with two enzymatically tailed samples were examined.

The polyA tails encoded in a DNA plasmid produced much narrower polyA tail distributions than mRNA that was enzymatically tailed post-IVT. In all cases, the enzymatically tailed mRNA produced significantly broader distributions of polyA tails than did plasmid-encoded polyA tails. See, FIG. 1. Enzymatically adenylated mRNAs with tail lengths that ranged from 30 A's to over 120 A's were also generated and analyzed. Our results agreed with those previously reported by Holtkamp et al., 2006. Blood 108: 4009-4017, who also found that enzymatically tailing resulted in a large distribution of tail lengths. Accordingly, and for the purpose of clarity, the inventors did all of our further work in the EXAMPLE using plasmid-encoded polyA tails.

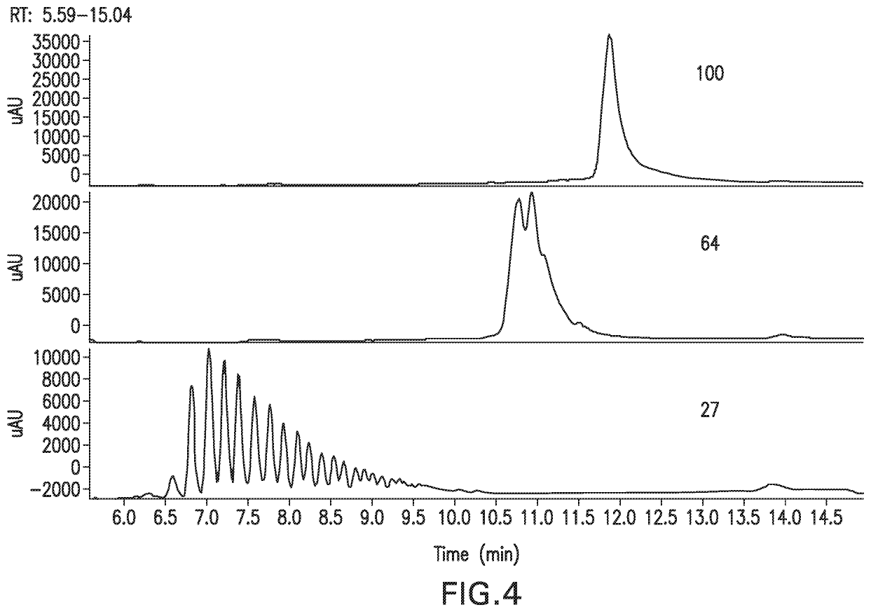

FIG. 4 shows that ion-paired reversed phase chromatographic resolution decreases with increasing tail length and single nucleotide resolution is lost between 27 (SEQ ID NO: 6) and 64 polyA tail length (SEQ ID NO: 11). Chromatographic separation of each distinct tail length is not needed for identification, because the mass spectra from co-eluting polyA species can be distinguished by the deconvolution software.

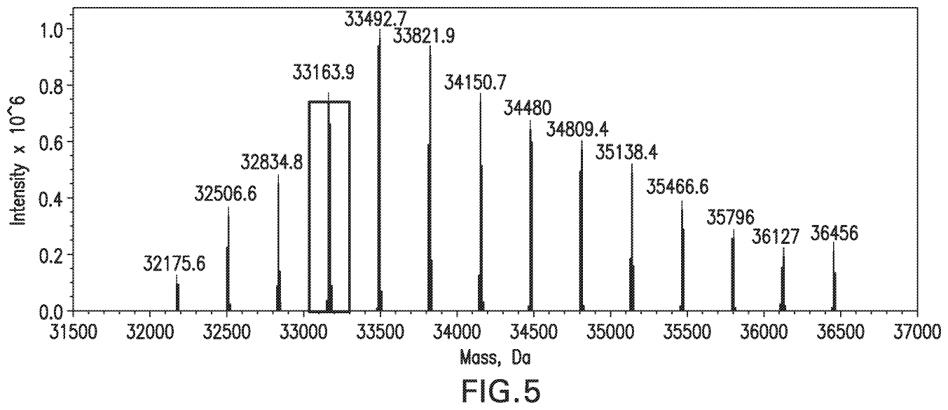

The processed or deconvoluted mass spectrum shown in FIG. 5 shows the mass for the expected 100 mer polyA tail (SEQ ID NO: 7) (mass 33,163 in box), along with a series of masses separated by 329amu (+1-2 amu), which is the mass of adenosine. Thus, the number of different polyA tail species and their relative abundance can be determined by the number and intensity of peaks separated by the mass of adenosine.

The observed masses are +/−2 amu from the theoretical 329 difference and this variance arises from the resolution of the mass spectrometer. However, because the next closest nucleotide in mass is guanosine (G), a 16amu difference is easily distinguishable. TABLE 1 shows the masses of the observed and expected T1 cleavage fragments for different tail lengths.

TABLE 1
Theoretical and experimentally observed masses
of various polyA tails after T1 digestion

| Tail length | Calculated Molecular | Observed Molecular |
|---|---|---|
| (CAₙ) | weight (avg) | weight |
| 117 | 39759.7 | 38760.6 |
| 100 | 33164.1 | 33163.9 |
| 64 | 21312.6 | 21312.2 |
| 27 | 9131.8 | 9130.8 |

PolyA Tail Length Distribution.

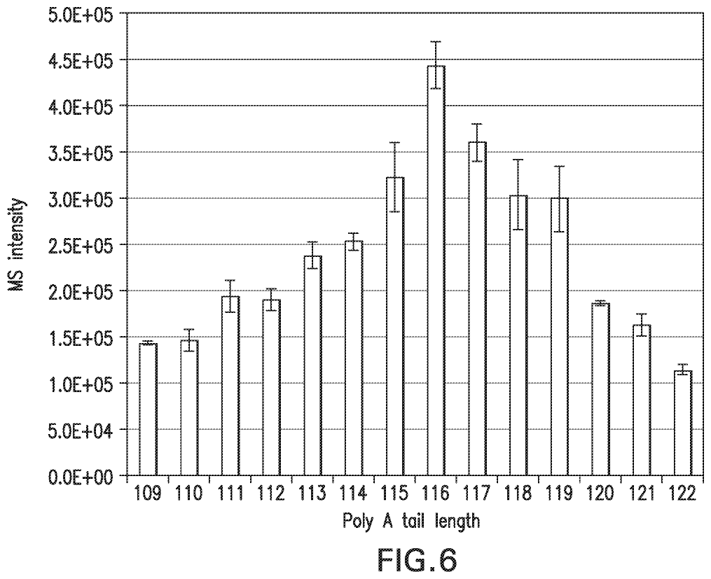

The reproducibility of the method of the invention was tested on a 2100nt mRNA with a polyA tail length of 117 (SEQ ID NO: 8). Four separate T1 digestions were analyzed. The resulting tail length distributions and the mass spectral intensities showed little variation. See, FIG. 6.

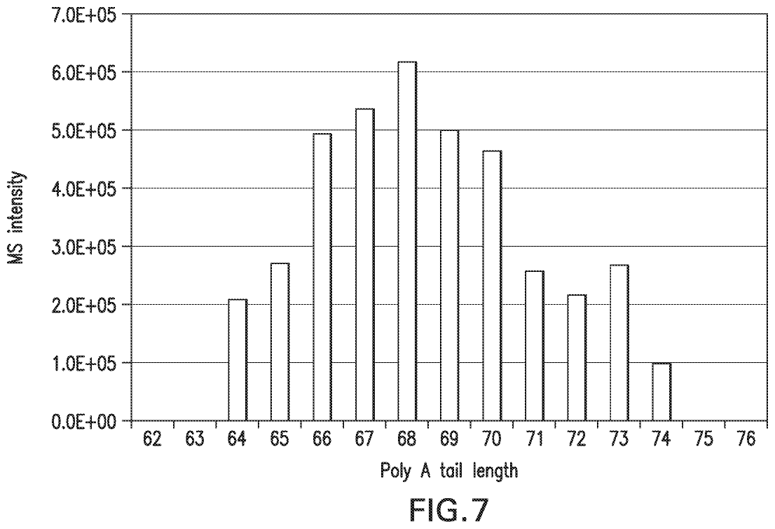

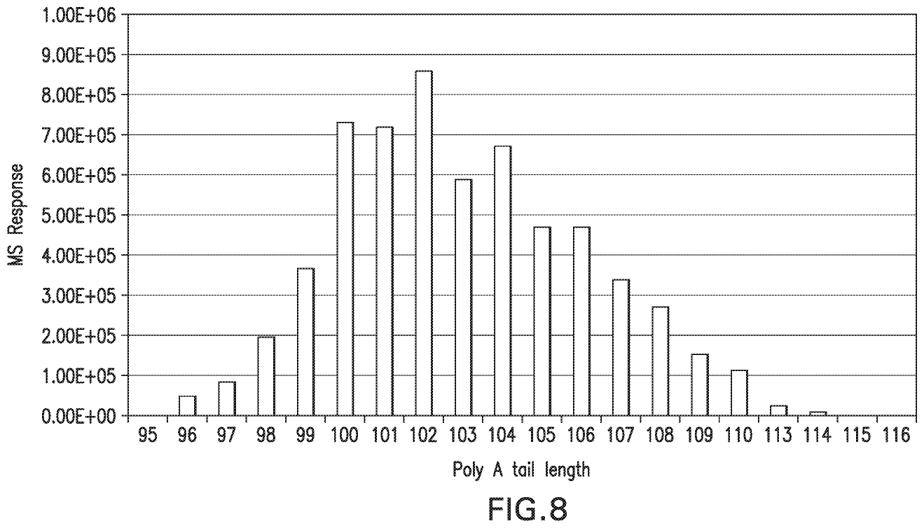

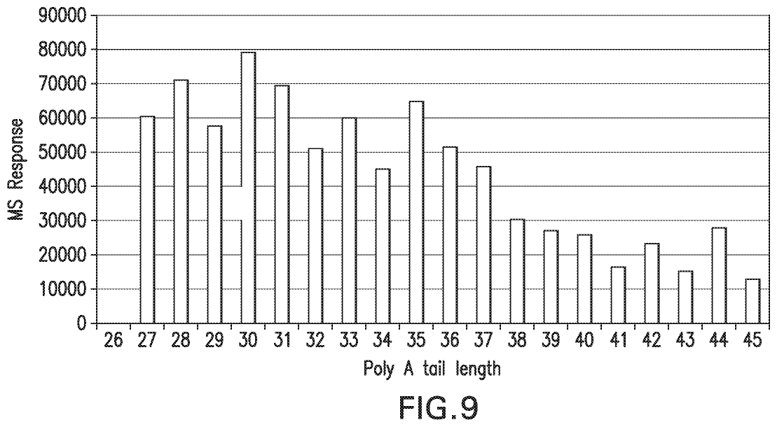

Our analysis of mRNAs with different polyA tail lengths showed that none of the mRNAs had a single unique tail length. Instead, each mRNA contained a distribution of lengths. See, FIG. 6 and FIG. 7. Closer inspection of our Sanger sequencing results showed populations of plasmids with different polyA tail lengths. For the plasmid encoding 117 A's (SEQ ID NO: 8), the fluorescent traces have a signal for adenosine ranging from position 109 to 122, despite being called at 117. Specifically, the Sanger sequencing results of the 3′ end of the plasmid used to synthesize the mRNA in FIG. 6, which showed fluorescent intensity traces for each base along with the called sequence, was called at having 117 A's (SEQ ID NO: 8). However, close inspection showed that other bases are also present, which decreases the number of A's in the tail to 109 and that additional A's are present out to 123. These minor populations were transcribed into the mRNA and the observed tail lengths closely matched the adenosine traces in the sequencing. This was also true for plasmids encoding 100 mer tails. See, FIG. 8. However, those encoding 27 mer tails contained tail lengths that were longer than expected from the plasmid. See, FIG. 9.

The 3′ poly A tail has been shown to affect mRNA function in a variety of ways and measurement of the tail length in IVT-synthesized mRNA can be helpful in understanding how length is related to function. Assessing the heterogeneity of poly A tails in IVT mRNA can also be important from a clinical standpoint as mRNA is increasingly being produced for therapeutic purposes and having a consistent and well-defined product is important to reproducible activity. In order to characterize IVT mRNA, we developed a simple and rapid LC-MS-based method for directly (no conversion to cDNA) determining poly A tail length with single-nucleotide resolution.

Tail lengths can be analyzed in less than 5 h with most of that time spent incubating for T1 cleavage. We have used magnetic oligo dT beads which are a common technique for isolating mRNA, and they worked well to capture cleaved poly A tails but did show a bias for capturing longer tails in a mixed population. This bias did not appear to be present for tails around 64 nt and above. Analysis of the isolated poly A tails employed standard LC-MS conditions for oligonucleo-tides and used routine MS data processing to calculate the masses of the tail species. The upper limit of the length of tail that can be analyzed, the resolution, and the sensitivity of that measurement are dependent on the mass spectrometer. As mentioned earner, sequences up to 500 nucleotides in length have been analyzed successfully so this is well within the reported range of mRNA poly A tail lengths. Using the theoretical mass to identify tail length does require knowledge of the 3′ sequence in order to calculate the mass of the T1 cleavage product which would prevent it from being used to determine the tail length of unknown mRNAs. Although even without sequence information, the distribution of tail lengths could be obtained from the number of peaks separated by the mass of a nucleotide.

Our first observations demonstrated that poly A tails encoded in the DNA plasmid produced much narrower poly A tail distributions than mRNA that was enzymatically tailed post-IVT. Using plasmid-encoded poly A tails of 27 (SEQ ID NO: 6), 64 (SEQ ID NO: 11), 100 (SEQ ID NO: 7), and 117 (SEQ ID NO: 8) in length, we found that the distribution of tail lengths closely matched the sequence populations observed in Sanger sequencing. However, when no variation in tail length was detected in the encoding plasmid, tails greater than the DNA template were observed. The variation in tail length unrelated to the plasmid sequence was attributed to transcriptional slippage by the RNAP. Different RNAPs did not alter tail length distributions and we found that T7, T3, and SP6 all produced the same amount of slippage. It is possible that mutated or other types of RNAPs could narrow the poly A tail distribution, for example it has been shown that human mitochondrial RNAP has less of tendency to slip than SP6.

The findings here indicate that tying a specifictail length to the amount of protein expression or other mRNA attribute is problematic particularly if enzymatic tailing is used. This could explain some of the variation between studies looking at optimizing poly A tail length and protein expression. For our specific system, however, we found that protein expression increased up to the maximum 117 poly A tail length (SEQ ID NO: 8) that was studied.

TABLE 2
SEQUENCE LISTING
Nucleotide Sequences

| | | SEQ |
|---|---|---|
| | | ID |
| Polynucleotide | Sequence | NO. |
| Cap0 | m7GpppGGGAGACGC | 1 |
| | GUGUUAAAUAACA | |
| T7 | GGATCCGGAGGCCG | 2 |
| polymerase | GAGAATTG | |
| promoter | | |
| upstream | | |
| enhancer | | |
| sequence | | |
| T7 | TAATACGACTCACTATA | 3 |
| Polymerase | | |
| Promoter | | |
| synthetic | CCUGAAAAAAAAAA | 4 |
| RNA | | |
| designed | | |
| to | | |
| produce a | | |
| 10mer | | |
| polyA | | |
| product | | |
| following | | |
| T1 | | |
| digestion | | |
| synthetic | CCUGAAAAAAAAAA | 5 |
| RNA | AAAAAAAAAA | |
| designed | | |
| to | | |
| produce a | | |
| 20mer | | |
| polyA | | |
| product | | |
| following | | |
| T1 | | |
| digestion | | |

The detailed description provided herein is to illustrate the invention, but not to limit its scope. Other variants of the invention will be readily apparent to one of ordinary skill in the biotechnological arts and are encompassed by the appended claims.

Each of the patents, patent publications, and patent applications, and all documents cited herein or during their prosecution (“application cited documents”) and all documents cited or referenced in the application cited documents, together with any instructions, descriptions, product specifications, and product sheets for any products mentioned therein or in any document therein and incorporated by reference herein, are hereby incorporated herein by reference, and can be used in the practice of the invention. All documents (e.g., these patents, patent publications and applications and the application cited documents) are hereby incorporated by reference.

The recitation of a listing of elements in any definition of a variable herein includes definitions of that variable as any single element, combination or sub-combination of listed elements. The recitation of an embodiment herein includes that embodiment as any single embodiment or in combination with any other embodiments or portions thereof.

While this invention has been disclosed with reference to specific embodiments, other embodiments and variations of this invention can be devised by one of skill in the biotechnological art without departing from the true spirit and scope of the invention. The appended claims are intended to be construed to include all such embodiments and equivalent variations.

1. A method of evaluating the quality of an mRNA composition, comprising the steps of:
providing an evaluation by mass spectrometry of the relative distribution of isolated polyA chain lengths or amount of a polyA chain length that have been cleaved from the mRNA of a sample obtained from the mRNA composition, to provide a test value and providing a determination of whether test value has an relative distribution or amount of a reference value, to thereby evaluate the quality of the mRNA composition.

2. The method of claim 1, wherein the method further comprises performing the mass spectrometry to determine the relative distribution of the isolated polyA chain lengths or the amount of a poly A chain length that have been cleaved from the mRNA of the sample.

3. The method of any of the preceding claim 2, wherein the mass spectrometry is LC-MS.

4. The method of any of the preceding claim 1, wherein the method further comprises providing a sample from the mRNA composition and cleaving the polyA tails from the mRNA in the sample using an enzyme or combination of enzymes that do not cleave adenosine.

5. The method of claim 4, wherein the enzyme is ribonuclease T1, RNAse CL3 (cusativin), RNase A or any combination thereof.

6. The method of claim 1, wherein the method further comprises isolating the cleaved polyA tails from the mRNA in the sample by hybridizing the cleaved polyA tails to a surface coated substrate conjugated with polynucleotides.

7. The method of claim 6, wherein the surface coated substrate is a magnetic bead.

8. The method of claim 7, wherein the magnetic bead is conjugated with oligo dT.

9. The method of any of the preceding claim 1, wherein the mRNA is made by in vitro transcription (IVT), e.g., by a method described herein.

10. A radiolabel-free method for analyzing the 3′-polyadenosine (polyA) tails of mRNA in an mRNA composition, comprising the steps of
(a) cleaving polyA tails from a sample of the RNA composition using ribonuclease T1, RNAse CL3 (cusativin), RNase A or any combination thereof;
(b) isolating the cleaved polyA tails by hybridization to surface coated substrate that are conjugated to a polynucleotide; and
(c) determining the relative distribution of poly A chain lengths or the amount of a polyA chain in the sample using mass spectrometry, to thereby analyze the polyA tails present in the mRNA composition.

11. The method of claim 10, wherein the mass spectrometry is LC-MS.

12. The method of claim 10, further comprising providing a test value based upon the relative distribution of the polyA chain lengths or amount of the polyA chain in the sample and comparing the test value to a reference value.

13. The method of claim 10, wherein the surface coated substrate comprises magnetic beads.

14. The method of claim 10, wherein the polynucleotide that is conjugated to the surface coated substrate is oligo dT.

15. The method of claim 10, wherein the mRNA is made by in vitro transcription (IVT).

16. (canceled)

17. The method of claim 10, wherein the polyA tails within the composition range from ˜20 A's to ˜200 A's in length.

18. A method of making an RNA composition, e.g., an mRNA composition, comprising:
providing an RNA sample from the RNA composition;
providing a relative distribution of polyA chain lengths or an amount of a polyA chain length from isolated polyA tails from the RNA in the RNA sample, by mass spectrometry, e.g., LC-MS or MALDI-MS, to provide a test value;
providing a determination of whether the test value is an amount or has a relative distribution of a reference value; and
further processing the RNA composition based upon the determination.

19. The method of claim 18, wherein the further processing is one or more of classifying, selecting, accepting or discarding, releasing or withholding, processing into a drug product, shipping, moving to a different location, formulating, labeling, packaging, releasing into commerce, or selling or offering for sale, based upon whether a preselected relationship between the test value and the reference value is met.

20. The method of claim 18, wherein the RNA sample has an amount of a polyA chain length or has a profile of polyA chain length distribution of a reference value, and the RNA composition is processed into drug product, formulated, labeled, packaged, or released into commerce based upon the determination.

21.-24. (canceled)

25. The method of claim 18, wherein the method further comprises cleaving the polyA tails from the mRNA in the sample using an enzyme or combination of enzymes that do not cleave adenosine.

26. The method of claim 25, wherein the enzyme is ribonuclease T1, RNAse CL3 (cusativin), RNase A or any combination thereof.

27. The method of claim 18, wherein the method further comprises isolating the cleaved polyA tails from the mRNA in the sample by hybridizing the cleaved polyA tails to a surface coated substrate conjugated with polynucleotides.

28. The method of claim 27, wherein the surface coated substrate is a magnetic bead.

29. The method of claim 28, wherein the magnetic bead is conjugated with oligo dT.

30. The method of claim 18, wherein the method further comprises producing the mRNA composition using IVT.

31. The method of claim 30, wherein the polyA tails of the mRNA composition are part of the DNA template for IVT.

32. The method of claim 30, wherein the poly A tails are enzymatically added to the IVT produced mRNA in the mRNA composition.

33. The method of claim 1, wherein the reference value is a value determined from an RNA sample from a commercially available RNA composition; a value determined from a previous batch of the RNA composition; a production standard imposed by a regulatory agency; or a release standard.

34.-36. (canceled)
